(19) United States
(12) Patent Application Publication
(10) Pub. No.: US 2006/0160837 A1
(43) Pub. Date: Jul. 20, 2006

(54) Rapamycin compounds in the treatment of neurofibromatosis type 1

(75) Inventor: Karen Cichowski, Newton, MA (US)

Correspondence Address: FITCH, EVEN, TABIN & FLANNERY, P. O. BOX 65973, WASHINGTON, DC, 20035, US

(73) Assignee: The Brigham and Women's Hospital, Inc., Boston, MA (US)

(21) Appl. No.: 11/319,124

(22) Filed: Dec. 28, 2005

Related U.S. Application Data
(60) Provisional application No. 60/639,776, filed on Dec. 29, 2004.

(51) Int. Cl. A61K 31/4745

(52) U.S. Cl. 514/291

(57) ABSTRACT

The present invention is directed to methods for treating neurofibromatosis type 1 (NF1) using rapamycin, derivatives of rapamycin or prodrugs of rapamycin. The invention covers the treatment both of non-malignant fibromas, as well as a variety of tumors associated with cells that have mutations in the NF1 gene.

### CROSS REFERENCE TO RELATED APPLICATIONS

The present application claims the benefit of, and priority to, U.S. provisional application No. 60/639,776 filed on Dec. 29, 2004.

### FIELD OF THE INVENTION

The present invention is directed to a method of treating neurofibromatosis type 1 (NF1) by administering rapamycin, a rapamycin derivative, or a rapamycin prodrug. For the most part, the NF1 patients will be treated for non-malignant neurofibromas. However, treatments may extend to malignant peripheral nerve sheath tumors, optic pathway gliomas, myeloproliferative disorders, and pheochromocytomas.

### BACKGROUND OF THE INVENTION

Neurofibromatosis type1 (NF1) (also known as von Recklinghausen NF or Peripheral NF), is a genetic disorder characterized by the growth of tumors along various types of nerves. Although the tumors are usually benign “neurofibromas,” they are often disfiguring, painful and, depending on location and size, can be debilitating. In addition, NF1 patients have a predisposition for the formation of myeloid malignancies, gliomas and pheochromocytomas. NF1 affects approximately 1 in every 3500 individuals world-wide (Stumpf, et al., Arch. Neurol. 45:575-578 (1988)) and, apart from palliative surgery, there is currently no effective treatment available.

NF1 is caused by mutations in the “NF1” gene (U.S. Pat. No. 5,605,799), many of which have been specifically identified (DeLuca, et al., Hum. Mutat. 23:629 (2004); Zou, et al., Oncogene 23:330-339 (2004); Orgine, et al., Hum. Mutat. 22:179-180 (2003); Baralle, et al., Am. J. Med. Genet. 119A:1-8 (2003); Kluwe, et al., Am. J. Med. Genet. 40:368-371 (2003); DeRaedt, et al., Am. J Hum. Genet. 22:1288-1292 (2003); DeLuca, et al., Hum. Mutat. 21:171-172 (2003); and Kluwe, et al., Hum. Mutat. 19:309 (2002)). This gene has been fully sequenced and identified as producing the tumor suppressor neurofibromin (U.S. Pat. Nos. 5,227,292; 6,238,861; Cawthon, et al., Cell 62:193-201 (1990); Vickochil, et al., Cell 62:187-192 (1990)). About half of the mutations are inherited from a parent and about half occur spontaneously in patients with no family history of the disease.

Rapamycin is a drug that has been marketed as an immunosuppressive agent to prevent organ or bone marrow rejection in transplant patients (Kessler, et al., Helv. Chim. Acta 76:117 (1993)). It is available from Wyeth-Ayerst Laboratories as a 1 mg/ml solution and in tablets of 1, 2 and 5 mg. Unlike other immunosuppressive agents, rapamycin does not appear to promote the development of malignancies (Guba, et al., Nature Med. 8:128-135 (1992)). In fact, studies suggest that it alters transcription or translation of multiple genes and that this leads to an inhibition of cellular growth. At present, rapamycin or its derivatives are being tested as treatment for several different cancers, including cancer of the prostate (van der Poel, et al., Urol. Res. 30:380-386 (2003); pancreas (Stephan, et al., Clin. Cancer Res. 10:6993-7000 (2000); kidney and (Luan, et al., Kidney Int. 63:917-926 (2003); lung (Boffa, et al., Clin. Cancer Res. 10:293-300 (2004)).

### SUMMARY OF THE INVENTION

The molecular target of rapamycin has been identified and is known as mTOR (mammalian Target of Rapamycin). mTOR has been shown to be critically involved in cell growth and proliferation. The present invention is based upon the discovery that cells isolated from benign and malignant tumors from NF1 patients (which therefore harbor a mutated NF1 gene), exhibit aberrant “activation” of the mTOR protein. Furthermore, rapamycin dramatically suppressed the proliferation and tumorigenic properties of these cells. Thus, the invention is based upon the concept that rapamycin is effective in treating the tumors and, particularly, the non-malignant neurofibromas, associated with NF1.

Thus, in its first aspect, the invention is directed to a method of treating a patient for neurofibromatosis type 1 by administering a therapeutically effective amount of a rapamycin compound. The term “therapeutically effective” indicates that a sufficient amount of drug is given to accomplish, at least in part, the therapeutic objective. In the case or the present claims, a sufficient amount of a rapamycin compound should be given to shrink the size of tumors or retard their formation or growth.

The term “rapamycin compound” refers to rapamycin, derivatives of rapamycin that have been described in the art which maintain the antitumor effect of rapamycin in NF1 patients, and prodrugs of rapamycin or rapamycin derivatives. The most preferred rapamycin compounds are rapamycin itself and the derivatives RAD001 (Novartis), AP23573 (Ariad) and CCI-779 (Wyeth).

The method described extends to the treatment of malignant tumors which have been identified as having cells exhibiting a mutation in the NF1 gene resulting in reduced neurofibromin activity. For example, tumor cells may be removed from a patient and examined for an NF1 mutation. If such a mutation is present, then treatment with a rapamycin compound would be initiated. Examples of tumors or conditions that can be treated include malignant peripheral nerve sheath tumors, malignant gliomas, astrocytomas and pheochromocytomas. In preferred embodiments, the rapamycin compound is rapamycin itself, RAD001, AP23573 or CCI-779 and is administered to patients at a dose of 0.1-500 mg per day, preferably at 1-50 mg per day and more preferably at 1-10 mg per day.

In all treatment methods, the rapamycin compound may be administered topically, typically in the form of a cream, lotion or ointment or given orally. Alternatively, it may be injected directly into a tumor or administered by an alternative route.

### DETAILED DESCRIPTION OF THE INVENTION

A. Rapamycin Compounds

Rapamycin is an immunosuppressive lactam macrolide which may either be purchased commercially (e.g., from A.G. Scientific, San Diego, Calif.) or synthesized using procedures that have been described in the art (Nicolaou, et al., J. Am. Chem. Soc. 115:4419-4420 (1993); Schreiber, J. Am. Chem. Soc. 115:7906-7907 (1993); Danisheffky, J. Am. Chem. Soc. 115:9345-9346 (1993)).

Rapamycin derivatives suitable for use in the invention are described in numerous references, including: WO 94/02136 (16-O-substituted derivatives); U.S. Pat. No. 5,258,389 (40-O-substituted derivatives); WO 94/9010 (O-aryl and O-alkyl derivatives); WO 92/05179 (carboxylic acid esters); U.S. Pat. Nos. 5,118,677 and 5,118,678 (amide esters); U.S. Pat. No. 5,118,678 (carbamates); U.S. Pat. No. 5,100,883 (fluorinated esters); U.S. Pat. No. 5,151,413 (acetals); U.S. Pat. No. 5,120,842 (silyl esters); WO 93,11130 (methylene derivatives); WO 94/02136 (methoxy derivatives); WO 94/02385 and WO 95/14023 (alkenyl derivatives); U.S. Pat. No. 5,256,790 (32-O-dihydro or substituted derivatives); EP 96/02441; U.S. 2004/023562 (carbohydrate derivatives); U.S. Pat. No. 4,316,885 (mono and diacylated derivatives); U.S. Pat. No. 5,120,725 (bicylic derivatives); U.S. Pat. No. 5,120,727 (rapamycin dimers); EP 467606 (27-oximes of rapamycin); U.S. Pat. No. 5,023,262 (42-oxo analogs); U.S. Pat. No. 5,177,203 (arylsulfonates and sulfamates); U.S. Pat. No. 5,177,203. In addition, various rapamycin prodrugs have been described in U.S. Pat. Nos. 4,650,803; 5,672,605; 5,583,189; 5,527,906; 5,457,111; 5,995,100; and 6,146,658. Of particular interest for use in treatment methods are derivatives described in patents owned by Novartis (U.S. Pat. Nos. 5,665,772; 5,912,253; 5,985,890; 5,912,253; 6,200,985; 6,384,046; and 6,440,990), Ariad (WO 96/41865); and Wyeth (U.S. Pat. Nos. 5,362,718; 6,399,625; 6,399,627; 6,432,973; 6,440,991; 6,677,357; and 6,680,718). The teachings of all of these patents and published applications are hereby incorporated by reference in their entirety.

It will be understood that the drugs described above may be administered to patients in any pharmaceutically acceptable form, for example, as a pharmaceutically acceptable salt. The most critical factor with respect to the compounds is their ability to inhibit TOR (target of rapamycin).

B. Drug Formulations

The present invention is compatible with the delivery of drugs by any means known in the art, including peroral, internal, pulmonary, rectal, nasal, vaginal, lingual, transdermal, intravenous, intraarterial, intramuscular, intraperitoneal, intra-tumoral, intracutaneous and subcutaneous routes. The most preferred routes are orally (especially using dosage forms such as tablets, capsules or solutions), topically, transdermally or by intra-tumoral injection. The amount of rapamycin compound present in a composition should, in general, be in the range of about 0.01 to about 30% w/w and preferably in an amount of 1 to 20% w/w.

Guidance in preparing formulations may be obtained from compositions of rapamycin that are commercially available and from descriptions in the art. Compositions may contain any of the standard inert components that are found in drug tablets, capsules, etc., including polymers; polyethylene glycol; cyclodextrins; saccharides; surfactants; disintegrants; antioxidants; stabilizers; flavoring agents; coloring agents, etc. Specific guidance for the preparation of a dosage form may be found in Remington's Pharmaceutical Sciences (1980) A. Oslo ed.

C. Treatment Methods

Patients diagnosed as having neurofibromatosis type 1 may be treated by administering one or more of the rapamycin derivatives and prodrugs described above. In general, patients will receive between about 1 mg per day and 10 mg per day. Naturally, these dosages can be adjusted by the attending physician based upon clinical conditions.

Tumors that may occur in patients not having neurofibromatosis may, nevertheless, be treated using rapamycin compounds, provided that they are dependent, at least in part, upon a loss of activity of the NF1 gene. Thus, to determine whether a given tumor will respond to rapamycin, one may remove cells from the tumor, e.g., at biopsy, and assay them to determine whether there are mutations in the NF1 gene, or whether there is an abnormally low level of the tumor suppressor neurofibromin. To test for the presence of mutations, standard techniques such as amplification of regions of the NF1 gene by PCR using primers based upon the known sequence of NF1 or blotting techniques may be employed. Tests for the presence of neurofibromin may take the form of ELISAs, radioimmunoassays or immunoblots.

Treatment involving the use of rapamycin compounds may be combined with other treatment methods to improve overall effectiveness. Once initiated, treatment should continue until tumors completely disappear or, alternatively, until tumor growth has been arrested.

### EXAMPLES

### I. Materials and Methods

Cell Culture and Immunoblots

Litter-mate matched MEFs or NIH3T3 cells were plated in serum-free media at a density of 1.0×10⁶ cells/10-cm plate. After 18 hours, 200 nM insulin or 6 μM LPA was added. Where indicated, cells were pre-treated with DMSO, 20 nM rapamycin (Calbiochem), 200 nM Wortmannin (Sigma-Aldrich), or indicated concentrations of U0126 (Calbiochem) for 30-min. For amino acid withdrawal studies, cells were washed and media was replaced with D-PBS for the indicated times. Following stimulation, cells were lysed in 1% SDS boiling lysis buffer. Neurofibroma-derived patient matched NF1^{+/-} and NF1^{-/-} Schwann cells were isolated and cultured as previously described (Rosenbaum, et al., J. Neurosci. Res. 61:524-532 (2000); Serra, et al., Hum. Mol. Genet. 9:3055-3064 (2000)). Schwann cells were seeded at a concentration of 2.5×10⁵ cells/6-cm plate in DMEM containing 0.1% serum without forskolin, insulin or heregulin and lysed as described above. Identical results were obtained in Schwann cell experiments that were acutely performed in the presence or absence of forskolin. Human MPNST cell lines were generated from NF1 patients as previously described (Frahm, et al., Neurobiol. Dis. 16:85-91 (2004)). First and second-hit mutations in the NF1 gene in tumor cells were verified by sequence analysis. Clarified lysates were normalized for protein levels and analyzed by Western blotting with the following antibodies: Phospho-p70S6K(T-389), Phospho-Tuberin (S-939), Phospho-Tuberin (T-1462), Phospho-Akt(S-473), Phospho-p44/42 Erk (T-202/Y-204) (Cell Signaling Technologies), Tuberin (C-20) (Santa Cruz Biotechnology), Protein Kinase Bα/AKT1, Actin, β-Tubulin (Sigma-Aldrich) and avian p90RSK1.

Retroviruses

Primary MEFs or NIH3T3 cells were infected with retrovirus expressing empty vector, human NF1 GRD (MSCV-GRD-pac), a dominant negative p90RSK (K112R), a dominant-negative p53 (pBabe-hygro-p53DD), or pLPC-E1A12S in the presence of 7.5-μg/ml polybrene. Cells were briefly selected in 2-μg/ml puromycin and/or 100-μg/ml hygromycin.

Ras Activation Analysis

NIH3T3 cells were plated in DMEM containing 3% serum at a density of 1.0×10⁶ cells/10-cm plate. After 18 hours, cell lysates were normalized and Ras-GTP was detected using a Ras-activation assay per manufacturer's protocol (Upstate Biotechnology).

Immunoprecipitations

Primary MEFs were harvested in Triton X-100 IP buffer (10 mM Tris pH 7.5, 50 mM NaCl, 50 mM NaF, 30 mM Na₄O₇P₂, 1.0% Triton X-100, Complete® protease inhibitor cocktail (Roche), 1 μM Microcystin-LR (Calbiochem), 100 nM Calyculin-A (Calbiochem)) and lysed on ice for 20-min. For MEFs, clarified lysates were normalized for protein concentration and incubated with anti-Tuberin (C-20) (1:100 dilution, Santa Cruz Biotechnology) where indicated. Immunoprecipitated proteins were resolved along with total cell extracts (10% of immunoprecipitation volume) via SDS-PAGE and immunoblots were performed with the indicated antibodies.

MPNST Proliferation Studies

MPNSTs were seeded at 6.5×10⁴ cells/6-cm well in normal growth medium containing rapamycin (0.01, 0.1, 1.0, 10, 100 nM) or equal volume of vector (DMSO). After 7 days, cells were trypsinized and live cells counted on triplicate plates using trypan blue exclusion.

Soft Agar Assays

Nf1^{-/-} and Nf1^{+/+} MEFs were infected with pLPC-E1A12S-puro and pBabe-p53DD-Hygro. 1.0×10⁴ selected MEFs were suspended in a DMEM containing 0.05% agar containing either rapamycin (0.1, 1.0, 10, 20 or 50 nM) or equal volume of vector (DMSO). Cells were seeded on a 0.34% agar base. Four to six weeks after the initial seed, colony growth was assayed by photographing and counting ten random fields of view per sample, in triplicate plates or wells. Colony size was assessed by using ImageJ software (v1.32J, NIH).

### II. Results

The mTOR Pathway is Aberrantly Activated in Nf1-Deficient Primary Cells

In higher eukaryotes mTOR is regulated by both growth factor and nutrient availability (Fingar, et al., Oncogene 23:3151-3171 (2004)). Growth factors have been shown to activate mTOR via a PI3 kinase dependent mechanism, whereas nutrients (amino acids) affect this pathway further downstream, at the level of tuberin and/or mTOR itself. Because neurofibromin negatively regulates the Ras/PI3 kinase pathway, we examined whether the mTOR pathway was deregulated in primary Nf1-deficient cells. The most commonly utilized in vivo readout of mTOR activation is the phosphorylation of a well-characterized substrate, S6 kinase (S6K1), at T-389. Phosphorylation at this site is dependent on mTOR and is required for maximal S6K1 activation. As expected, serum starved wild-type mouse embryonic fibroblasts (MEFs) exhibited little activation of AKT or S6K1. However, AKT was aberrantly activated in serum deprived Nf1-null MEFs. Notably, S6K1 was also hyper-phosphorylated in these cells in the absence of any growth factors. The aberrant activation of S6K1 was inhibited by rapamycin, demonstrating its dependence on mTOR.

We also examined whether amino acid deprivation would inhibit mTOR activation in Nf1-deficient cells. Consistent with the observation that nutrients integrate into the mTOR pathway downstream of PI3 kinase activation, amino acid withdrawal resulted in the appropriate termination of the mTOR signal in both wild-type and Nf1-deficient cells. Thus, neurofibromin-deficiency specifically results in the deregulation of the mTOR pathway in response to growth factor deprivation.

mTOR Activation is Dependent on Ras

Because neurofibromin is a RasGAP we investigated whether the aberrant activation of the mTOR pathway in Nf1-deficient cells was dependent on the inappropriate activation of Ras. Notably, we and others have previously shown that Ras-GTP levels are deregulated in Nf1-deficient cells (Kim, et al., Oncogene 11:325-335 (1995); Bollag, et al., Nat. Genet. 12:144-148 (1996); Cichowski, et al., Genes & Development 17:449-454 (2003)). We therefore sought to determine whether expression of only the catalytic, GAP-related domain (GRD) of neurofibromin would be sufficient to suppress the observed defect in S6K1 activation. Importantly, expression of this domain resulted in a dramatic decrease in Ras-GTP levels and suppressed the activation of Ras effector pathways as has been reported by Hiatt and colleagues (Hiatt, et al., J. Biol. Chem. 276:7240-7245 (2000)). Moreover, in Nf1-deficient cells it restored the level of phospho-S6K1 to levels equivalent to those observed in wild-type cells under the same conditions, indicating that the hyper-activation of this pathway in Nf1-deficient cells is dependent on the absence of its RasGAP activity, rather than an additional uncharacterized function of neurofibromin.

Growth factor-induced mTOR activation is known to be dependent on PI3 kinase activity. However, growth factor receptors activate PI3 kinase via both Ras-dependent and independent mechanisms. Currently, it is unclear whether Ras is required for maximal mTOR activation in the context of normal growth factor signaling. Because expression of the GRD of neurofibromin dramatically inhibited RasGTP levels in wild-type cells, this reagent was used to determine whether Ras activation was required for maximal activation of the mTOR pathway in response to growth factors. Importantly, the GRD significantly attenuated AKT activation and blunted S6K1 activation in response to LPA and insulin. Interestingly, in both cases the GRD appeared to have an effect on AKT and S6K1 at later time points, rather than immediately after growth factor treatment. This may suggest that Ras-independent mechanisms of PI3 kinase activation are important for the initial activation of this pathway, while Ras is required for a more sustained activation. This model is consistent with the fact that Ras-independent activation of PI3 kinase occurs more proximal to receptor activation than Ras-dependent activation of its effectors, in both receptor tyrosine kinase and G protein-coupled receptor signaling. Regardless, these data indicate that Ras activation is required for maximal activation of the mTOR pathway in response to growth factors.

Hyper-Activation of the mTOR Pathway in Nf1-Deficient Cells is Dependent on Both PI3K and MEK Activation

To determine whether the inappropriate activation of mTOR in Nf1-deficient cells was solely dependent on the Ras/PI3 kinase effector pathway, we examined S6K1 phosphorylation in Nf1-deficient cells in the presence of the PI3 kinase inhibitor wortmannin, or the MEK inhibitor, U0126. As anticipated, wortmannin dramatically reduced S6K1 phosphorylation in serum-deprived Nf1-deficient cells. Surprisingly however, the MEK inhibitor suppressed S6K1 phosphorylation in these cells to a similar extent. These results suggest that the aberrant activation of mTOR in Nf1-deficient cells is dependent on both the Ras/PI3 kinase and Ras/Raf/MEK effector pathways.

To investigate how PI3 kinase and MEK might both contribute to the inappropriate activation of mTOR observed in Nf1-deficient cells, we examined the potential involvement of the TSC2 gene product, tuberin, in these cells. The primary target of AKT in this pathway is thought to be tuberin, which is phosphorylated at two distinct sites, T-1462 and S-939, by this kinase. AKT phosphorylation of tuberin has been shown to inactivate the TSC1/TSC2 complex, resulting in the subsequent activation of Rheb and mTOR, through an unknown mechanism. Importantly, in Nf1-deficient serum starved cells tuberin was constitutively phosphorylated at T-1462 and S-939, in contrast to wild-type cells where phosphorylation was minimal under these conditions. Phosphorylation at these sites was suppressed in the presence of wortmannin; however, the MEK inhibitor had no effect, suggesting that the loss of neurofibromin affects tuberin phosphorylation at these sites exclusively via the PI3K/AKT pathway.

Interestingly, a third regulatory phosphorylation site on tuberin has recently been reported. Blenis and colleagues have shown that PMA induces tuberin phosphorylation at S-1798 via the activation of the MEK/ERK/RSK pathway. This site was shown to be phosphorylated by an activated RSK1 in vivo and in vitro, and phosphorylation was blocked by a dominant negative RSK1 construct in vivo, suggesting that RSK1 can function as a tuberin kinase. Importantly, an alanine substitution at this site (S-1798) significantly reduced S6K1 activation in response to PMA and insulin. Furthermore, this mutation cooperated with T-1462A and S-939A mutations to inhibit tuberin function, suggesting that all three sites may be critical under certain conditions. Presumably, the loss of neurofibromin might also effect tuberin phosphorylation at this site via its effects on the Ras/Raf/MEK pathway. To test this hypothesis we examined tuberin phosphorylation at S-1798 in Nf1-deficient cells. This phosphorylation site is recognized by a phospho-specific antibody that recognizes a RXRXXpS/T consensus sequence. Blenis and colleagues have shown that this is the primary site activated by the MEK/ERK/RSK pathway and the phosphorylation site recognized by this antibody is blocked by dominant negative RSK1. We found that in Nf1-deficient serum starved MEFs, immunoprecipitated tuberin is hyperphosphorylated at this basophilic site. Importantly, phosphorylation at this site is suppressed by U0126 and is unaffected by wortmannin.

Because RSK1 is thought to mediate phosphorylation at this site, Nf1-deficient cells were infected with a retrovirus that expresses a dominant negative RSK1 protein. Importantly, expression of the DN-RSK1 suppressed the aberrant phosphorylation at this site in Nf1-deficient cells. Moreover, it reduced S6K1 phosphorylation to levels observed in wild-type cells. Taken altogether these results suggest that the loss of neurofibromin results in the inactivation of tuberin via both the Ras/PI3K/AKT and Ras/Raf/MEK/ERK/RSK pathways.

NF1-Deficient Human Tumor Cells Exhibit an Activated mTOR Pathway and are Hypersensitive to Rapamycin

To determine if NF1-deficient tumors from NF1 patients similarly exhibit an aberrant activation of the mTOR pathway, we examined this pathway in NF1+/− and NF1−/− Schwann cells derived from human neurofibromas. Neurofibromas are known to be extremely heterogenous lesions. Based on genetic studies of human tumors as well as mouse modeling data, it is believed that neurofibromas develop in NF1 patients as a result of “second hit” mutations in Schwann cells or Schwann cell precursors, which then act as a seed population to recruit other cells (NF1+/− Schwann cells, fibroblasts, mast cells, perineurial cells) into a developing lesion. Methods for isolating Schwann cells and then further separating NF1 +/− Schwann cells from NF1 −/− Schwann cells have been well described. We obtained matched NF1+/− and NF1−/− Schwann cells from one cutaneous and one plexiform neurofibroma, derived from two different patients. In both cases S6K1, AKT, and tuberin phosphorylation was significantly higher in the NF1- null cells as compared to the matched NF1 +/− cells. Furthermore, S6K1 phosphorylation was blocked by both wortmannin and U0126 in mutant cells, indicating that both the PI3K and MEK pathway contribute to activation of mTOR in these tumor cells as well.

It is commonly believed that tumors become dependent on the dysregulation of a specific signaling pathway and are therefore hypersensitive to its down-regulation. To determine if NF1-associated peripheral nerve sheath tumors have become dependent on hyper-activation of the mTOR pathway, we tested the effects of rapamycin on tumors from NF1 patients. For this experiment we utilized malignant tumors (MPNSTs), which arise from benign neurofibromas. We found that the proliferation of two independently derived MPNST cell lines from two NF1 patients was dramatically suppressed at low concentrations of rapamycin. The IC₅₀ for these experiments was between 1-10 nM. Notably, these IC₅₀ values are comparable to or lower than the IC₅₀ values of rapamycin derivatives that were tested on PTEN—deficient tumors, in which activation of the mTOR pathway has been well characterized. In addition, in one of the two cell lines, higher concentrations of rapamycin induced cell death.

As an independent means of assessing the effects of rapamycin on the tumorigenic properties of NF1-deficient cells, we established a genetically engineered cell system. Cellular transformation is known to require multiple genetic events affecting the Rb, p53 and Ras pathways (Hahn, Nat. Rev. Cancer 2:331-341 (2002)). In MEFs, the combined expression of a dominant-negative p53 gene and the E1A oncogene, which binds and inactivates Rb family members, is not sufficient to promote growth in soft agar. However we found that in the absence of Nf1, cells expressing these genes did form colonies in soft agar. Importantly, rapamycin suppressed colony growth at low concentrations in this system as well. Taken together, these results suggest that NF1-deficient tumor cell lines are exquisitely sensitive to rapamycin and suggest the potential therapeutic utility of rapamycin or its derivatives in treating tumors in NF1 patients.

### III. Discussion

The NF1 tumor suppressor was shown to function as a RasGAP shortly after the gene was cloned in 1990. Accordingly, elevated levels of Ras-GTP are observed in NF1-deficient tumors and cells. However, to date, the Ras-effector pathways that are responsible for disease pathogenesis have not been defined. We show here that the mTOR pathway is critically deregulated in NF1-deficient primary cells and human tumors. Importantly, tumor cells are highly sensitive to the mTOR inhibitor rapamycin, suggesting that it or its derivatives may be useful therapeutically. To date there is no effective treatment or cure for NF1. Neurofibromas, while benign, can be extremely problematic. In addition to a high tumor burden encumbering some patients, many cannot be surgically resected because of underlying nerve involvement. Furthermore, lesions that are surgically reduced typically re-grow. Therefore, the suggestion that deregulation of the mTOR pathway participates in NF1-related tumorigenesis may represent a therapeutic breakthrough for this disease. Notably, NF1 is quite prevalent: greater than 10 times more prevalent than most other phakomatotic disorders, and even more common than TSC, a disease for which the effects of rapamycin are being assessed in clinical trials.

While the activation of mTOR is known to be PI3 kinase-dependent, the extent to which Ras participates in this process is unknown. In fact, because growth factor receptors can activate PI3 kinase via a Ras-independent mechanism, Ras is generally excluded from discussions of the mTOR pathway entirely (Raught, et al., Proc. Nat'l Acad. Sci. USA 98:7037-7044 (2001); Hay, et al., Genes Dev. 18:1926-1945 (2004)). Notably, the over-expression of an activated Ras allele can activate S6K1. However it has been unclear whether the activation of endogenous Ras might similarly feed into this signaling pathway, in a tumorigenic setting, or in the context of normal growth factor signaling. By using Nf1-deficient cells we were able to uniquely address this question. We found that the activation of endogenous Ras, via the loss of neurofibromin, results in pathogenic levels of mTOR activation. Furthermore, by over-expressing the catalytic domain of neurofibromin, we showed that in wild-type cells Ras activation is required for maximal mTOR activation in response to growth factors. While this may be a subtle point, the amplitude and duration of Ras effector pathways has been shown to be critical for specifying biological responses (Marshall, Cell 80:179-185 (1995)). Therefore, it is possible that threshold levels of mTOR activation are not achieved in the absence of a Ras signal, which may have important biological consequences in certain settings. Regardless, these data demonstrate that Ras does function to amplify this signal.

Interestingly, we found that both the PI3 kinase pathway and the MEK pathway are required for the observed hyper-activation of mTOR in NF1-deficient cells and tumors. Specifically, these signals converge to phosphorylate tuberin at distinct sites. Importantly, a dominant-negative RSK1 blocks tuberin phosphorylation at the MEK dependent site, and completely suppresses S6K1 activation in Nf1-deficient cells, suggesting that RSK1 is the kinase that mediates this critical event. These results are particularly interesting in the context of other recent findings. First, it has been reported that tsc regulates photoreceptor differentiation downstream or in parallel to Ras/MAPK in Drosophila (Bateman, et al., Cell 119:87-96 (2004)). A second report has also implicated the ERK effector RSK1 in phosphorylating tuberin at S-1798, which participates in its inactivation downstream of PMA (Roux, et al., Proc. Nat'l Acad. Sci. USA 101:13489-13494 (2004)). We have gone on to show that this phosphorylation event occurs in NF1-deficient cells, is mediated by Ras, MEK and RSK1, and that this signal is absolutely required for the aberrant activation of mTOR in these cells. It remains to be determined if this phosphorylation event is generally required for tuberin inactivation. Given that NF1-mutations result in the aberrant activation of the PI3K/AKT pathway, it would have been expected that this signal would be sufficient to induce mTOR activation, as is thought for PTEN-deficient cells and tumors. However, the potential involvement of the MEK pathway in PTEN-deficient cells has never formally been tested. In PTEN-deficient tumors, it is possible that a cooperating mutation in the Ras/Raf pathway, or a signal emanating from growth factors (that engages the Raf/MEK pathway), might be required for maximal mTOR activation. Alternatively, known cross-talk between the PI3K and ERK pathways may also participate in this process and may occur in primary PTEN-deficient cells as well. Regardless, both pathways are required in NF1-deficient cells and human tumors.

Finally, rapamycin derivatives have been suggested as potential therapeutic agents for a variety of cancers (Luo, et al,, Cancer Cell 4:257-262 (2003); Sawyers, Cancer Cell 4:343-348 (2003)). The basis for this suggestion is that multiple genes are mutated in tumors, therefore inhibition of more than one pathway may be required for a therapeutic effect. On this basis neurofibromatosis type I may represent a uniquely treatable disease. Notably, benign symptoms, such as neurofibromas, can be quite severe. However, they are likely to occur from the mutation of a single gene: NF1. Therefore it is possible that rapamycin derivatives may represent a single hit therapy for these lesions, either by preventing tumor development and/or promoting their regression. The fact that a subset of these lesions are dermal, may also make them uniquely accessible to non-systemic modes of delivery, such as topical treatment. In any case, our data suggest that these agents may represent one of the first viable therapies for NF1.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A method of treating a patient for neurofibromatosis type 1 (NF1) comprising: administering to said patient a therapeutically effective amount of a rapamycin compound.

2. The method of claim 1, wherein said rapamycin compound is rapamycin or a rapamycin derivative selected from the group consisting of: RAD001; AP23573 and CCI-779.

3. The method of claim 1, wherein said rapamycin compound is administered to said patient at a dosage of 0.1-50 mg per day.

4. The method of claim 3, wherein said rapamycin compound is administered to said patient at a dosage of 1-10 mg per day.

5. The method of claim 4, wherein said rapamycin compound is administered topically or by intra-tumor injection to said patient.

6. A method of treating a patient for a malignant or nonmalignant tumor, wherein said tumor comprises cells having one or more mutations in the NF1 gene, comprising administering to said patient a therapeutically effective amount of a rapamycin compound.

7. The method of claim 6, wherein said rapamycin compound is rapamycin or a rapamycin derivative selected from the group consisting of: RAD001; AP23573 and CCI-779.

8. The method of claim 6, wherein said rapamycin compound is administered to said patient at a dosage of 0.1-50 mg per day.

9. The method of claim 8, wherein said rapamycin compound is administered to said patient at a dosage of 1-10 mg per day.

10. The method of claim 8, wherein said rapamycin compound is administered topically or by intra-tumor injection to said patient.

11. A method of treating a patient for a malignant peripheral nerve sheath tumor, wherein said tumor comprises cells having one or more mutations in the NF1 gene, comprising administering to said patient a therapeutically effective amount of a rapamycin compound.

12. The method of claim 11, wherein said rapamycin compound is rapamycin itself or a rapamycin derivative selected from the group consisting of: RAD001; AP23573; and CCI-779.

13. The method of claim 11, wherein said rapamycin compound is administered to said patient at a dosage of 0.1-50 mg per day.

14. The method of claim 13, wherein said rapamycin compound is administered to said patient at a dosage of 1-10 mg per day.

15. The method of claim 13, wherein said rapamycin compound is administered topically or by intratumor injection to said patient.

16. A method of treating a patient for a glioma, astrocytoma, myeloproliferative disorder or pheochromocytoma, wherein said glioma, astrocytoma, myeloproliferative disorder or pheochromocytoma have one or more mutations in a NF1 gene that reduces the activity of neurofibromin.

17. The method of claim 16, wherein said rapamycin compound is rapamycin itself or a rapamycin derivative selected from the group consisting of RAD001; AP23573; and CCI-779.

18. The method of claim 16, wherein said rapamycin compound is administered to said patient at a dosage of 0.1-50 mg per day.

19. The method of claim 18, wherein said rapamycin compound is administered to said patient at a dosage of 0.5-10 mg per day.

20. The method of claim 18, wherein said rapamycin compound is administered topically or intra-tumorally to said patient.
